SEQUENCE LISTING (14 sequences)

SEQ ID NO: 1 - DNA, 2487 bp, Saccharomyces cerevisiae
  CDS (1)..(2484)
atgctgtttggagtgaagttggctaatgaggtttatcctccttggaagggttcttatattaattatgagggcttgaaaaaattcctgaaggaagatagcgtgaaggatggaagtaacgataagaaagcacgctgggacgattcagatgaatccaagtttgtggaagagttggataaggaacttgaaaaagtctatggttttcaactaaaaaagtacaataacttgatggagagattgtcccatctggagaaacaaacagatacggaagcagccataaaggccttggacgctgatgcattccaacgtgtattggaggaactgttaagcgagtctaccgaattagacaattttaagagattgaactttactgggtttgctaagattgttaagaaacatgacaagctatatccaaagtatccatctgttaaatctttgttggaagttagattaaaggaattgccttcccattcggaagaatattccccattgttgtatcgtatttcatttttgtacaacattttgagaagtaattttaacactgcatctgaacccttagccagcgcttctaagttttctagcattgtcagcaatgacatagacatgaatttcagaagctttaaattttgggttcataatgacaacttaatggaggtcaaaacaagaatcttgagacatcttcccgtgttggtctacgccaatgttccctccgaaaacgatgacctggtcaatagattcgaatcagatatatcaaataatgatgaaattgtgggtagttcgagctccactagtagcgtagaacatggcttgggagcgcgctccttcgatccattaatcaacacgctatattttgacaatgagcattttgaattatataacgacaagttattaaagttaaattcagcacctactttaagattaaggtggactggccagttatctgataagccggatattttcttggaaaagaaaactcttattgaagacgaagccactgggaagtctgaatttgatctaactaaattgcaattgaaacaaaaattcattaacgggtttattttcgaaggtgataagaaatttaaagaacaaactttgaaaaagttaaaagaaagtggtacggcagggagagacctggaaaggttagaagaagatttctctgagattcaaaactttattatcaagaatgaattgcaaccagtttttagaactgtttacaccagaactgctttccagattcccggcgatgacaagataagagtaaccattgactctaatattgtattcatcaaggaggattcattcgacagagagcgtccaattagagaccctaatacctggcatagaactgatattgatgccaatgttgcaaatcccttgaaattcctgagaggaggcgagtatgctaagtttccttattcagtaatggaaattaaagtgaaaagttcattagattcttcgatgtctgccagttctatgatttctaatgtaaaactgcctaaaaagcatggtcaatggctgaacgatttgacaaattctcatttggtcaaagaaattccaaagttttctatctttgtgcaaggtgtggcatcattgtatggagatgatgaaaaattagatatcttaccattttggttaccagatttggaaacagatattagacaggatcctaagcaagcatatgaggaggaaaagaaaaaactgttgaaacaaaaagagatacaaaagaaaattgatggaatgagaaggctttccaacttaaaagagcctcaacatcaagcagcagtaccggtatctcaagaggaaaatgagcgtattacctctcaaggtgatttggaggcagacggttcatccgatgaggaaactgagcaagaacctcattcgaaaagatcaaagaaagttcggagaagaaaacccaaggccactttcttgagaattttggccggtagagatccaaagttaatgggggtggattctgaagaagaagaaattgaattgccacctggtgtgaaaaaaccattaaatttgttaaaaaatgctggtcctgtaaacgtggaggcaaaggtttggcttgccaacgaacgtacatttaacagatggttaagtgtcaccagtttattgagtgttttgacgttctcaatttataattctgtgaagaaagccgaataccccactttggctaactacatggcatacgtatattttggtctaacgatattctgtgctttatggtcctattccatttatatgaaaagagttgatattattcaacaaagaagcggtcaacatctagatgcaccacttggtccagttttggtttctatagttttatttgtcactttagtggttaattttgttatggcgtttagaaatgcagcaaagtctcgtcaagagttgcaaatacagaatttagaagttcctgaaagaataccagaagtattaaggccacttcaaaattatctattcaagttaatggggccaagcagtgattag

SEQ ID NO: 2 - protein, 828 aa, Saccharomyces cerevisiae
MLFGVKLANEVYPPWKGSYINYEGLKKFLKEDSVKDGSNDKKARWDDSDESKFVEELDKELEKVYGFQLKKYNNLMERLSHLEKQTDTEAAIKALDADAFQRVLEELLSESTELDNFKRLNFTGFAKIVKKHDKLYPKYPSVKSLLEVRLKELPSHSEEYSPLLYRISFLYNILRSNFNTASEPLASASKFSSIVSNDIDMNFRSFKFWVHNDNLMEVKTRILRHLPVLVYANVPSENDDLVNRFESDISNNDEIVGSSSSTSSVEHGLGARSFDPLINTLYFDNEHFELYNDKLLKLNSAPTLRLRWTGQLSDKPDIFLEKKTLIEDEATGKSEFDLTKLQLKQKFINGFIFEGDKKFKEQTLKKLKESGTAGRDLERLEEDFSEIQNFIIKNELQPVFRTVYTRTAFQIPGDDKIRVTIDSNIVFIKEDSFDRERPIRDPNTWHRTDIDANVANPLKFLRGGEYAKFPYSVMEIKVKSSLDSSMSASSMISNVKLPKKHGQWLNDLTNSHLVKEIPKFSIFVQGVASLYGDDEKLDILPFWLPDLETDIRQDPKQAYEEEKKKLLKQKEIQKKIDGMRRLSNLKEPQHQAAVPVSQEENERITSQGDLEADGSSDEETEQEPHSKRSKKVRRRKPKATFLRILAGRDPKLMGVDSEEEEIELPPGVKKPLNLLKNAGPVNVEAKVWLANERTFNRWLSVTSLLSVLTFSIYNSVKKAEYPTLANYMAYVYFGLTIFCALWSYSIYMKRVDIIQQRSGQHLDAPLGPVLVSIVLFVTLVVNFVMAFRNAAKSRQELQIQNLEVPERIPEVLRPLQNYLFKLMGPSSD

SEQ ID NO: 3 - DNA, 2508 bp, Saccharomyces cerevisiae
  CDS (1)..(2505)
atgctatttggtattaaactggctaacgacgtatatcctccctggaaggattcatatatcgactatgaaaggttgaagaaattactaaaggaaagcgtcatacatgatggccgtagttccgtggatagttggtccgaaaggaatgaatctgattttgtggaggccttggataaagaactagaaaaggtttacacatttcaaatttccaaatataatgctgtcttgcgaaaattagatgatttagaggaaaacactaaatcagctgaaaaaatccaaaaaataaattctgagcagttcaaaaacaccttagaagaatgtctagatgaggctcaaagattggataactttgacaggttaaattttactgggtttatcaagattgtgaagaaacacgataaattacatccaaactatccttctgtaaagtctcttttacaagtcagattgaaagaacttcctttcaacaattcagaagagtattctcccttgttgtataggatctcatatctgtacgagttcttgagatcaaattatgatcatccaaatacggtgtctaaatcattagcaagtacttctaaattatcacatttttctaaccttgaagacgcaagttttaaaagctacaagttttgggttcatgatgataatataatggaggttaaggctagaatcttaaggcatctacctgctttggtttatgcgtcggttccaaatgaaaatgacgatttcgtcgataatctagaatcagatgtccgcgtacaacctgaagcgcgcttgaatattggttcaaagagtaacagcctttctagtgatggtaatagcaaccaagatgttgaaattggaaaatcaaagagcgttattttcccacagtcatatgatccaacaatcactacattatatttcgacaacgatttttttgatttgtacaataacagattgttgaaaatcagcggtgcacctacgctgagattaagatggatcggtaaactgctagacaaacctgacatatttttggaaaagagaacttttacagaaaataccgaaactggtaattcaagttttgaagaaatcagattgcaaatgaaagccaaattcatcaacaattttatttttaaaaatgatcccagttacaaaaactatttaattaatcaactaagagaaaggggtactcaaaaggaggaactggaaaaactttctagagactttgataacatccaaaactttattgttgaggagaagttgcagccagttttgagagctacctataatagaactgctttccaaattcctggagatcaaagtattagagtcactatcgattccaatatcatgtacattagagaggattctttggacaaaaataggcccattaggaaccctgagaattggcaccgtgacgatattgattccaatattcccaacccattgaggtttttaagagcaggagaatattcgaagttcccctattctgtaatggagattaaggttataaaccaagataattctcaaatgcctaattatgagtggattaaagatttaactaattcacatttagttaacgaagttccaaaattttctttgtacttgcaaggtgtggcttcactgtttggtgaagacgataaatatgtcaacattttgccattctggttgcccgatttagaaaccgacatcagaaagaaccctcaggaggcttacgaagaagagaagaaaactttacaaaaacaaaagagcattcatgataaacttgataatatgagaaggttatccaaaatctctgtaccagatggaaagaccactgaaagacaagggcaaaaagatcagaatactcgccacgttattgcagatttagaagatcacgaatcatcagatgaagaaggtactgcattgcccaagaaatctgcagtcaaaaaagggaagaaattcaaaacgaatgcagctttcttgaaaattcttgctggaaaaaatatttcagaaaatggaaatgatccatactccgacgatacagacagtgcctcttctttccaattacctccgggagttaaaaaaccagttcaccttttgaaaaatgccggtcctgtcaaagttgaggcaaaagtttggctcgccaatgaacgtacattcaatagatggttaagtgtaaccacattgctgagtgtattgaccttttctatctataattcagtgcaaaaagccgaatttccacaactagctgatctgttggcctatgtatatttctttttgactttgttttgcggagtatgggcttatagaacctacttaaaaagattaactcttattaaaggtagaagtggtaagcatttggatgcacctgtgggacctattttggttgcagttgtattaatcgttaccttggttgttaactttagtgtggcttttaaagaggccgctaggagggaaagaggattagtaaacgtttcctcccagccttcgttaccccgtacactaaaaccaattcaagattttatcttcaatttggttggggaataa

SEQ ID NO: 4 - protein, 835 aa, Saccharomyces cerevisiae
MLFGIKLANDVYPPWKDSYIDYERLKKLLKESVIHDGRSSVDSWSERNESDFVEALDKELEKVYTFQISKYNAVLRKLDDLEENTKSAEKIQKINSEQFKNTLEECLDEAQRLDNFDRLNFTGFIKIVKKHDKLHPNYPSVKSLLQVRLKELPFNNSEEYSPLLYRISYLYEFLRSNYDHPNTVSKSLASTSKLSHFSNLEDASFKSYKFWVHDDNIMEVKARILRHLPALVYASVPNENDDFVDNLESDVRVQPEARLNIGSKSNSLSSDGNSNQDVEIGKSKSVIFPQSYDPTITTLYFDNDFFDLYNNRLLKISGAPTLRLRWIGKLLDKPDIFLEKRTFTENTETGNSSFEEIRLQMKAKFINNFIFKNDPSYKNYLINQLRERGTQKEELEKLSRDFDNIQNFIVEEKLQPVLRATYNRTAFQIPGDQSIRVTIDSNIMYIREDSLDKNRPIRNPENWHRDDIDSNIPNPLRFLRAGEYSKFPYSVMEIKVINQDNSQMPNYEWIKDLTNSHLVNEVPKFSLYLQGVASLFGEDDKYVNILPFWLPDLETDIRKNPQEAYEEEKKTLQKQKSIHDKLDNMRRLSKISVPDGKTTERQGQKDQNTRHVIADLEDHESSDEEGTALPKKSAVKKGKKFKTNAAFLKILAGKNISENGNDPYSDDTDSASSFQLPPGVKKPVHLLKNAGPVKVEAKVWLANERTFNRWLSVTTLLSVLTFSIYNSVQKAEFPQLADLLAYVYFFLTLFCGVWAYRTYLKRLTLIKGRSGKHLDAPVGPILVAVVLIVTLVVNFSVAFKEAARRERGLVNVSSQPSLPRTLKPIQDFIFNLVGE

SEQ ID NO: 5 - DNA, 1947 bp, Saccharomyces cerevisiae
  CDS (1)..(1944)
atgaagtttggtgagcacttgagcaagtctcttattagacagtacagctactattatattagttatgatgatctgaagactgagctagaagataacttatctaagaataacggtcagtggacgcaagaattggaaacagattttttagaatctttggagatcgagttagacaaggtttacacattttgcaaagttaagcatagtgaagtttttagacgtgtaaaagaggttcaagaacaagtgcaacatactgttcgtttgttagactccaataatcctcctactcagctagattttgagattctagaagaagaactaagtgatatcattgccgatgttcatgatctagcaaagttttcgagactaaactacactggtttccaaaagattatcaagaaacacgataagaagacaggttttatcttgaaaccagttttccaagttagattagactctaaaccatttttcaaggaaaactatgacgaactagtcgttaaaatttcccaactatatgatattgccagaacttcaggtcgcccaatcaagggagactcatctgctggtgggaagcaacaaaatttcgttagacagacaacaaagtattgggttcaccctgataacattacagaattgaagctgatcatcttgaagcatttaccagtgttagtcttcaacactaataaggaattcgaaagagaagattccgctattacttcgatctattttgataatgaaaatctagacctttattatggcagattaagaaaagatgaaggtgcagaagcccacagattgagatggtatgggggtatgtctacagacacgatctttgtagaaagaaagacccatagagaggattggactggtgagaaatctgtcaaggcaagatttgcgctaaaggaacgtcatgttaatgacttcttaaagggtaaatatactgttgatcaagtatttgccaagatgcgtaaagaaggcaagaagccaatgaacgaaattgaaaacttggaggcgttagcatccgaaattcaatacgtcatgttaaagaaaaagttgagacctgtagtgagatcattctacaatagaactgcttttcaattacctggtgatgcaagagttcgtatctcccttgatacagagttaactatggtgagagaagacaactttgatggtgtggatagaactcataagaattggaggagaactgatattggtgtcgattggccattcaagcagctagatgacaaggatatttgccgtttcccatatgcagtcctggaagttaaattgcaaactcaactaggtcaagaacctccagagtgggtacgtgaattagtcgggtctcacttagttgagccagtcccaaaattctccaagtttattcatggtgtggctaccttactaaatgataaagtggattcaatcccattttggttacctcaaatggatgttgatatcaggaaacctccactaccaacaaatattgaaataacaagacccggtagatccgataacgaagataacgacttcgatgaggatgatgaggatgacgctgctttggttgctgccatgacaaacgcacccggtaattctcttgatatagaagaatctgttggttacggggctacttcggcccccacctccaacactaatcatgttgttgagagtgctaatgctgcttattatcaaagaaagatcaggaatgccgaaaatcctatctccaagaaatactatgaaatcgtggcattttttgaccactattttaacggtgatcaaatatctaaaatcccaaaaggtactacttttgatactcaaattcgtgccccaccggaaagacgatatgtgtgccagttcgtgtggaaccaaaagtttactttgccactgaaagaacctacctgtcttggttgtccatctcgatattgttgggcggtgtctccactactttattaa

SEQ ID NO: 6 - protein, 648 aa, Saccharomyces cerevisiae
MKFGEHLSKSLIRQYSYYYISYDDLKTELEDNLSKNNGQWTQELETDFLESLEIELDKVYTFCKVKHSEVFRRVKEVQEQVQHTVRLLDSNNPPTQLDFEILEEELSDIIADVHDLAKFSRLNYTGFQKIIKKHDKKTGFILKPVFQVRLDSKPFFKENYDELVVKISQLYDIARTSGRPIKGDSSAGGKQQNFVRQTTKYWVHPDNITELKLIILKHLPVLVFNTNKEFEREDSAITSIYFDNENLDLYYGRLRKDEGAEAHRLRWYGGMSTDTIFVERKTHREDWTGEKSVKARFALKERHVNDFLKGKYTVDQVFAKMRKEGKKPMNEIENLEALASEIQYVMLKKKLRPVVRSFYNRTAFQLPGDARVRISLDTELTMVREDNFDGVDRTHKNWRRTDIGVDWPFKQLDDKDICRFPYAVLEVKLQTQLGQEPPEWVRELVGSHLVEPVPKFSKFIHGVATLLNDKVDSIPFWLPQMDVDIRKPPLPTNIEITRPGRSDNEDNDFDEDDEDDAALVAAMTNAPGNSLDIEESVGYGATSAPTSNTNHVVESANAAYYQRKIRNAENPISKKYYEIVAFFDHYFNGDQISKIPKGTTFDTQIRAPPERRYVCQFVWNQKFTLPLKEPTCLGCPSRYCWAVSPLLY

SEQ ID NO: 7 - DNA, 390 bp, Saccharomyces cerevisiae
  CDS (1)..(387)
atgtcttcagcaccattattacaaagaacacctgggaaaaagatcgctttgcccacacgagttgagccaaaagtgttctttgccaatgagcgtacctttttgtcgtggttgaactttacagttatgctgggaggccttggtgtaggtttactgaattttggtgacaagataggtagggtcagtgcaggactatttacttttgttgccatgggtacaatgatatacgcgcttgtaacataccactggagagctgctgcgattagacgtagaggatcaggtccttatgatgacagattggggcccactttgttgtgctttttcttattggttgctgtcattatcaactttatattaagattgaagtacaatgacgctaacactaagttatga

SEQ ID NO: 8 - protein, 129 aa, Saccharomyces cerevisiae
MSSAPLLQRTPGKKIALPTRVEPKVFFANERTFLSWLNFTVMLGGLGVGLLNFGDKIGRVSAGLFTFVAMGTMIYALVTYHWRAAAIRRRGSGPYDDRLGPTLLCFFLLVAVIINFILRLKYNDANTKL

SEQ ID NO: 9 - DNA, 2025 bp, Saccharomyces cerevisiae
  CDS (1)..(2022)
atggttgtagtgggtaagagtgaagtgcgcaatgtaagcatgagtaggccgaagaagaaatcattgattgccatcctgtcgacctgtgttttgtttttcctcgtgtttataattggtgctaaatttcaatatgtttccgttttctccaaattcttagatgacaggggggacaacgagtcacttcaattgctcaatgacatagaatttacaaggctaggactgacacctagggagccagtaattatcaaagacgtaaagacagggaaggagagaaaattacatggacggttccttcatattaccgatattcatcctgacccttattatgtggaaggaagctctattgatgcagtttgtcacacaggaaaaccaagcaaaaaaaaggatgtggcacccaagtttgggaaggcaatgtctggatgtgattctcccgttattttaatggaagaaactctgagatggattaaagaaaatcttagagataagattgattttgtcatttggacgggtgataatatcagacatgacaatgaccggaaacatccaagaacagaagcacaaatttttgatatgaacaatatcgttgctgataaaatgacagaattatttagtgctggcaatgaagaagatccaagagattttgatgtgtctgtcattccaagtcttggaaacaatgatgtctttccacataacatgtttgcactaggaccaactctacaaactagagaatattataggatttggaaaaattttgttccgcagcagcagcaaagaacttttgataggagtgcttcatttttgactgaagttattccagggaagcttgctgtcctgtcaattaacacgctgtacttattcaaggccaatccgttagttgacaattgtaattcaaaaaaagaaccaggttaccaacttttactttggttcggttatgtattagaagaactaaggagtaggggaatgaaagtatggttaagtggacatgtacctccaatcgcaaaaaatttcgatcagtcgtgctacgataagttcacgctatggactcacgaatacagggatataatcattggaggattatacggtcacatgaatattgaccacttcatcccaacggatggtaaaaaagctagaaaatcgttattgaaagccatggagcaatccactcgtgttcaacaaggagaagacagtaatgaagaggacgaagaaactgaattgaatagaattctggatcacgctatggcggcaaaagaagtttttttaatgggagcgaaaccatctaacaaagaagcatatatgaacaccgtccgcgacacatactatcggaaagtgtggaataagctggaaagagtggatgaaaaaaatgttgaaaatgagaagaaaaagaaagaaaagaaggacaaaaaaaagaaaaagccaatcactagaaaagagctcattgaacgctattccattgtgaacataggtggttcagtcattccaactttcaatccttcctttaggatctgggaatataacatcaccgacatagtgaatgactccaattttgcagtttcggagtataagccctgggatgaatttttcgagtcactaaataagattatggaagactctttgctagaagatgaaatggacagcagtaatatcgaggttgggatcaaccgcgagaagatgggcgaaaagaaaaacaagaagaaaaagaaaaatgacaagactatgccaattgagatgccagacaaatacgaactcggccctgcatatgtaccgcagttattcactccgacgcgtttcgtccaattctacgccgacttggaaaaaatcaatcaagaattacataattcatttgttgaatctaaggatattttcaggtatgagatagaatatacctcagacgagaaaccatactcaatggattcgttaacagtaggaagttatttggatcttgcaggcagattatacgaaaacaaacctgcatgggaaaaatacgtcgaatggtcatttgcgtcttctggatataaagatgattaa

SEQ ID NO: 10 - protein, 674 aa, Saccharomyces cerevisiae
MVVVGKSEVRNVSMSRPKKKSLIAILSTCVLFFLVFIIGAKFQYVSVFSKFLDDRGDNESLQLLNDIEFTRLGLTPREPVIIKDVKTGKERKLHGRFLHITDIHPDPYYVEGSSIDAVCHTGKPSKKKDVAPKFGKAMSGCDSPVILMEETLRWIKENLRDKIDFVIWTGDNIRHDNDRKHPRTEAQIFDMNNIVADKMTELFSAGNEEDPRDFDVSVIPSLGNNDVFPHNMFALGPTLQTREYYRIWKNFVPQQQQRTFDRSASFLTEVIPGKLAVLSINTLYLFKANPLVDNCNSKKEPGYQLLLWFGYVLEELRSRGMKVWLSGHVPPIAKNFDQSCYDKFTLWTHEYRDIIIGGLYGHMNIDHFIPTDGKKARKSLLKAMEQSTRVQQGEDSNEEDEETELNRILDHAMAAKEVFLMGAKPSNKEAYMNTVRDTYYRKVWNKLERVDEKNVENEKKKKEKKDKKKKKPITRKELIERYSIVNIGGSVIPTFNPSFRIWEYNITDIVNDSNFAVSEYKPWDEFFESLNKIMEDSLLEDEMDSSNIEVGINREKMGEKKNKKKKKNDKTMPIEMPDKYELGPAYVPQLFTPTRFVQFYADLEKINQELHNSFVESKDIFRYEIEYTSDEKPYSMDSLTVGSYLDLAGRLYENKPAWEKYVEWSFASSGYKDD

SEQ ID NO: 11 - DNA, 405 bp, Rattus rattus
  CDS (1)..(402)
atggccgagcagtcagacaaggatgtgaagtactacactctggaggagattcagaagcacaaagacagcaagagcacctgggtgatcctacatcataaggtgtacgatctgaccaagtttctcgaagagcatcctggtggggaagaagtcctaagagagcaagctgggggtgatgctactgagaactttgaggacgtcgggcactctacggatgcacgagaactgtccaaaacatacatcatcggggagctccatccagatgacagatcaaagatagccaagccttcggaaacccttatcactactgtcgagtctaattccagttggtggaccaactgggtgatcccagccatctcagccctggtggtagctctgatgtatcgcctctacatggcagaagattaa

SEQ ID NO: 12 - protein, 134 aa, Rattus rattus
MAEQSDKDVKYYTLEEIQKHKDSKSTWVILHHKVYDLTKFLEEHPGGEEVLREQAGGDATENFEDVGHSTDARELSKTYIIGELHPDDRSKIAKPSETLITTVESNSSWWTNWVIPAISALVVALMYRLYMAED

SEQ ID NO: 13 - DNA, 363 bp, Saccharomyces cerevisiae
  CDS (1)..(360)
atgcctaaagtttacagttaccaagaagttgccgaacacaatggcccagaaaatttctggattatcatcgatgacaaagtttacgatgtttctcaattcaaagatgaacatccaggtggtgatgaaattataatggatttgggtggacaagatgctacagaaagctttgtcgatatcggtcattctgacgaagcattgagactactgaaaggtttatacattggtgacgttgacaagaccagtgagcgcgtttctgtggaaaaggtatctacctctgaaaaccaaagtaaaggtagtggtacattggttgtcatattggccattttaatgctaggtgttgcttattatttgttgaacgaataa

SEQ ID NO: 14 - protein, 120 aa, Saccharomyces cerevisiae
MPKVYSYQEVAEHNGPENFWIIIDDKVYDVSQFKDEHPGGDEIIMDLGGQDATESFVDIGHSDEALRLLKGLYIGDVDKTSERVSVEKVSTSENQSKGSGTLVVILAILMLGVAYYLLNE

(19) United States
(12) Patent Application Publication
(10) Pub. No.: US 2005/0244822 A1
(43) Pub. Date: Nov. 3, 2005

(54) Method of monitoring gene expression

(76) Inventors: Tetsuro Kokubo, Kanagawa (JP); Masahiro Shirakawa, Kanagawa (JP); Jeremy Robin Howard Tame, Kanagawa (JP)

Correspondence Address: MORGAN & FINNEGAN, L.L.P., 3 WORLD FINANCIAL CENTER, NEW YORK, NY, 10281-2101, US

(21) Appl. No.: 10/501,039

(22) PCT Filed: Jan. 9, 2003
(86) PCT No.: PCT/JP03/00117

§ 371 Date: Jun. 23, 2005

(30) Foreign Application Priority Data
Jan. 9, 2002 (JP) 2002-002396

(51) Int. Cl. C12Q 1/68

(52) U.S. Cl. 435/6

(57) ABSTRACT

The present invention provides a technique by which highly detailed gene expression profiling data for analyzing the gene function can be obtained safely and conveniently. It is a versatile non-destructive and high-resolution visualizing technique by which expression levels of genes can be monitored in real time, and which can be applied in vivo and to deep tissues. For instance, plasmids in which molecules that vary the NMR signal and can be quantified by PHM genes are incorporated downstream from promoters responding to the condition of medium, external stimuli and the like, are introduced into the cells. The level of polyphosphate in deep tissues and the like can then be quantified by ³¹P-NMR, allowing expression profiling of the promoter, both non-destructively and in real time.

### Technical Field

The present invention relates to a non-destructive method of monitoring gene expression using NMR and a method for screening various types of agents by using the method of monitoring.

### Background Art

Understanding at the molecular level what regulates gene expression in eukaryotes is one of the most important research projects in contemporary biology. Approximately 20 years have passed since R. Roeder succeeded in demonstrating in vitro transcription for the first time, numerous experiments using in vitro transcription systems have been performed all over the world, and a number of findings relating to the respective elements including the purification/cloning of general transcription factor TFIID by the present inventors have been obtained in the meantime. However, when these findings are applied to biological systems, inexplicable results are still frequently observed. It is presumed that this is not because respective elements are scattered, but because many elements function together as a system in vivo. It is thought that in vitro experimental systems alone cannot model this behavior, and development of better in vivo experimental systems is essential in order to appreciate these regulatory systems.

β-galactosidase and luciferase, which require exogenous substrates, or GFP, which is autofluorescent, have been developed as reporter genes and they have brought useful data at various phases conventionally. The applications for these proteins have been generally limited to transparent organisms, and they have not been useful in the case of opaque samples such as deep tissues of animals. On the other hand, in opaque samples, a method in which transferrin receptor and β-galactosidase are used as reporter genes for high sensitivity imaging by magnetic resonance (MRI) of exogenous genes has been known, but delivery of specific substrate is necessary in both cases. For instance, the former requires fusion of microparticles including iron oxide (called MION) with transferrin, while the latter requires a gadolinium-ion complex (called EgadMe) containing a galactopyranosyl ring. Further, once these substrates are accumulated in vivo they are not degraded easily, therefore they are not suitable for monitoring the expression level of genes in real time. Moreover, it is realistically impossible to make numerous tissue sections, in order to examine gene expression of the whole genome (including as much as 30,000 genes in the case of higher eukaryotes) on a cellular level at very high resolution, and reconstruct it on computer. Apart from a few exceptional organisms having transparent bodies, such as Brachydanio rerio (zebrafish) and Caenorhabditis elegans, gene expression can only be examined in detail by preexisting methods such as in situ hybridization of thin sections. However, as the expression of most of the genes involved in development of the central nervous system is switched on or off extremely rapidly, even analysis with tissue sections is an extremely difficult task. Accordingly, the technique of whole body micro-imaging is becoming increasingly important.

Although the genome sequences of many organisms have now been determined along with the progress of Genome Project, the expression profile of a gene is crucial in understanding the gene function. It is not widely known that promoter region is essential in specifying the temporal and spatial control of gene transcription, and therefore provides extremely valuable data for analyzing the function of proteins encoded downstream. Although it is natural that several genes relating to the same phenomena are expressed in the same cells, there are few cases in which these kinds of data are actually used in discovering the function. This is because the expression of very few genes has been determined at a cellular level of detail. Consequently, it is essential in comprehending the entire biological system to collect the expression data of as many genes as possible, and construct the database of expression profile hence.

As previously mentioned, in opaque samples such as deep tissues of animals, the method of measuring by MRI using transferrin receptors and β-galactosidase genes is known, but both reporters require specific substrates, which are accumulated in vivo and degraded slowly, therefore they are not suitable for monitoring expression level of gene in real time, further it is extremely difficult in practice to distribute substrates to all the cells evenly. Besides, non-destructive methods of quantifying that can be applied to deep tissues, μPET (micro-positron emission tomography), SPECT (single-photon emission computed tomography) and the like are known, but their spatial resolution is around one millimetre, and insufficient for visualizing a single cell. The development of a versatile visualizing technique is therefore necessary in vivo.

The subject of the present invention is to provide a technique by which the most important gene expression profiling data for analyzing gene function can be obtained safely and conveniently, i. e. a non-destructive and high-resolution visualization in vivo technique with high versatility, by which expression levels of genes can be monitored in real time, and which can be applied to deep tissues and the like.

Promoter structure is important in determining the transcription initiation site. When the genes on genomes are transcribed, it is thought that multistep responses that cannot be reproduced in vitro, such as structural changes within the nucleus, chromosome, template DNA and the like occur successively. In the meantime, though Saccharomyces cerevisiae is a simple unicellular organism having only 6,000 genes, it comprises transcription machinery that is approximately equivalent to that of Man, and it is currently the most attractive model organism to elucidate the detailed pattern of gene expression from the perspective of data expression system. A group of genes (PHM genes) relating to the polyphosphate synthetic pathway was identified in Saccharomyces cerevisiae recently for the first time as eukaryotes (Molecular Biology of the Cell, Vol. 11, 4309-4321, 2000).

In Saccharomyces cerevisiae, it is known that along with the decrease of concentration of phosphate in the medium, transcription factor PHO4 shifts to a hypophosphorylated form from a hyperphosphorylated form. As a result, the intranuclear level of PHO4 increases to activate a set of target genes relating to phosphate metabolism. Though polyphosphate synthetases PHM1-4 were isolated as candidates for target genes of PHO4, analysis showed that these encode membrane proteins which are very similar to each other, and the level of polyphosphate decreases in each of the deleted strains. Accumulation of polyphosphate cannot be seen at all in phm1Δphm2Δdouble mutation line, phm3Δline and phm4Δline, which strongly suggests that PHM1-4 genes encode the subunits of polyphosphate synthetase, which is thought to function in vacuoles or on vacuolar membranes. The present inventors considered that the aforementioned polyphosphate synthetase (PHM) gene of Saccharomyces cerevisiae might be able to be used as a reporter gene, by quantifying polyphosphate, which is known to be significantly accumulated in all organisms. This quantitation can be achieved by ³¹P-NMR directly, or indirectly by measuring the influence on nearby water of iron ions binding to polyphosphate by ¹H-NMR. Plasmids were constructed carrying PHM genes downstream of promoters responding to various conditions (e.g. environmental stimuli), and introduced into a PHM deleted strain of Saccharomyces cerevisiae. The intracellular expression level of polyphosphate was quantified by ³¹P-NMR and ¹H-NMR non-destructively and in real time, showing that the intracellular level of polyphosphate in the yeast cells varies depending on the expression level of the PHM genes. The present invention has thus completed.

### Disclosure of the Invention

The present invention relates to a method of monitoring the expression of a chosen gene, wherein accumulation of a molecule that varies a NMR signal and can be quantified by NMR, without the requirement to add an exogenous substrate (“1”); the method of monitoring expression of a chosen gene according to “1”, wherein the molecule that varies the NMR signal and can be quantified by NMR is a polyphosphate (“2”); the method of monitoring expression of a chosen gene according to “2”, wherein the polyphosphate is a polyphosphate generated by expression of a polyphosphate synthetase gene placed downstream of the chosen gene (“3”); the method of monitoring expression of a chosen gene according to “2”, wherein the polyphosphate is a polyphosphate generated by expression of the polyphosphate synthetase gene placed downstream of the chosen gene in-frame (“4”); the method of monitoring expression of a chosen gene according to “1”, wherein the molecule that varies the NMR signal and can be quantified by NMR is a type of cytochrome (“5”); the method of monitoring expression of a chosen gene according to any one of “1”to “5”, wherein the quantification by NMR is a non-destructive quantification by NMR (“6”); the method of monitoring expression of a chosen gene according to any one of “1”to “6”, wherein the expression of the chosen gene is monitored in real time (“7”); the method of monitoring expression of a chosen gene according to any one of “1”to “7”, wherein the expression level of the chosen gene within a cell, a tissue or an organ is detected (“8”); and the method of monitoring expression of a chosen gene according to any one of “1”to “8”, wherein the chosen gene is a target gene of a general transcription factor (“9”).

The present invention further relates to a method for screening various types of agents, wherein the method of monitoring expression of a chosen gene according to any one of “1”to “9” is used (“10”).

### BRIEF DESCRIPTION OF THE DRAWINGS

FIG. 1 shows a picture indicating the result of quantifying phosphate (³¹p) NMR spectra in cell populations of wild-type and phm4 knockout (ΔPHM4) Saccharomyces cerevisiae (S. cerevisiae).

FIG. 2 shows a picture indicating the result of one-dimensional imaging of cells from wild-type Saccharomyces cerevisiae (S. cerevisiae) by ³¹P-NMR.

FIG. 3 shows a picture indicating the result of quantifying the amount of polyphosphate in cell population of phm4 knockout line (ΔPHM4 (gal1-10PHM4) in which plasmids carrying PHM4 genes downstream of the GAL1 promoter were transformed, wild-type line and phm4 knockout line (ΔPHM4) of S.cerevisiae, using the same method as that used in FIG. 1

FIG. 4 shows a picture indicating the result of analyzing colonies of wild-type (upper panel) and phm4 knockout (lower panel) Saccharomyces cerevisiae, grown on YPD media containing 1 mM FeCl₃, by ¹H-NMR.

### BEST MODE FOR CARRYING OUT THE INVENTION

The present invention includes any methods of monitoring in which expression is quantified by NMR and no-exogenous substrate is added to the cells. For this purpose, molecules that can be detected by NMR through their effects on the signals can be found from those that absorb radio-frequency fields because of the presences of magnetic moments, which are products of nuclear orbital moments and spin moments, intrinsic to their nucleus. In addition to poly-phosphates, cytochromes and ferritins, both of which contain iron-heme groups, are good examples.

As mentioned above, polyphosphate can accumulate in all organisms in significant amounts and can be quantified directly by ³¹P-NMR. Polyphosphate synthetase genes, for instance PHM genes of Saccharomyces cerevisiae, specifically PHM 1-4 genes placed downstream of the chosen gene and in-frame with it allow transcription to be quantified in real time. The polyphosphate generated by this method has a mean length of up to 50 phosphate groups, within the detectable range by NMR. The 10 mer is thought to show the highest sensitivity for ³¹P-NMR. The polymer sizes can be confirmed by staining the cell contents with toluidine blue after polyacrylamide gel-electrophoresis. Importantly, NMR signals derived from polyphosphate can be independently quantified without interference from signals of nucleic acids. DNA sequences of PHM 1-5 genes and amino acid sequences of PHM 1-5 are shown by Seq. I.D. Nos. 1-10. As for the polyphosphate synthetases used, there is no particular restriction as long as the synthetases can generate polyphosphate intracellularly. PPK (polyphosphate kinase) derived from prokaryotic organisms, functional homologues, or orthlogues, other than PHM of the aforementioned Saccharomyces cerevisiae can be used.

As for using cytochromes molecules, in addition to cytochromes b/c₁/c/a₃, cytochrome c oxidase, cytochrome P450, cytochrome b-559, and b-563 can be used but cytochrome b-5 is preferable since its signal is readily measured by NMR. The DNA sequence of cytochrome b-5 gene from rat, the amino acid sequence of the protein, the DNA sequence of the cytochrome b-5 gene from yeast, and the amino acid sequence of the protein are shown by Seq. I.D. No. 11, Seq. I.D. No. 12, Seq. I.D. No. 13, and Seq. I.D. No. 14, respectively for reference. Apart from heme proteins having iron atoms such as, ferritin, hemoglobin, catalase, peroxidase and the like can also be used as a reporter gene, placed downstream in-frame with the chosen genes.

As for which genes may be studied by the method of monitoring described by the present invention, there are no particular restrictions. Target genes of general transcription factors such as TFIID subunit (TAF), and the like, i.e. genes which are targeted by general transcription factors and regulated their expression by general transcription factors for instance RPS5 gene, HIS4 gene, TUB2 gene and the like are suitable examples for which expression profiling data may be determined by the invention described.

The present invention is particularly suitable for real-time non-destructively monitoring of expression of the chosen gene in opaque samples like deep tissues of animals, so as to detect expression level of the chosen genes in cells, tissues or organs. In order to perform monitoring of expression of the chosen gene according the present invention, the PPK (polyphosphate kinase) gene from Escherichia coli may be placed downstream of the chosen promoters and the amount of polyphosphate accumulated can be quantified by NMR. Thus PPK may be used a reporter instead of the aforementioned Saccharomyces cerevisiae PHM genes. Escherichia coli PPK can synthesize polyphosphate from ATP in a single step, unlike the synthetic system of eukaryotic cells. Other polyphosphate synthetic systems from animals can be used as reporter genes.

As for the method for screening various types of agents that regulate the expression of the chosen gene, it is not particularly restricted as long as it is a method for screening using the aforementioned method of monitoring gene expression by NMR. For instance, by bringing cells or tissues either expressing or accumulating molecules that vary NMR signals and which are detectable by NMR into contact with a test substance in vitro, quantifying the expression level of the chosen genes by NMR, and comparing/evaluating the expression level with control agents, without test agents, it is possible to screen for substances inhibiting or promoting the expression of the proteins which are translation products of the chosen genes. As for such candidate agents, therapeutic agents against AIDS, leukemia, viral carcinogenesis such as cancer and the like can be exemplified.

As for the NMR apparatus used in the method of monitoring an expression of the chosen gene and the method for screening the various types of agents of the present invention, it is not particularly restricted as long as it varies NMR and can quantify the NMR signal to be measured. Preferably the probe sections will enable high resolution and the bore will be large. NMR apparatus with good magnetic field gradient amplifiers will allow the method of the invention to be applied not only to microorganisms but also to small animals and plants.

The present invention will be explained more specifically with examples below, but the technical scope of the invention is not limited to these examples.

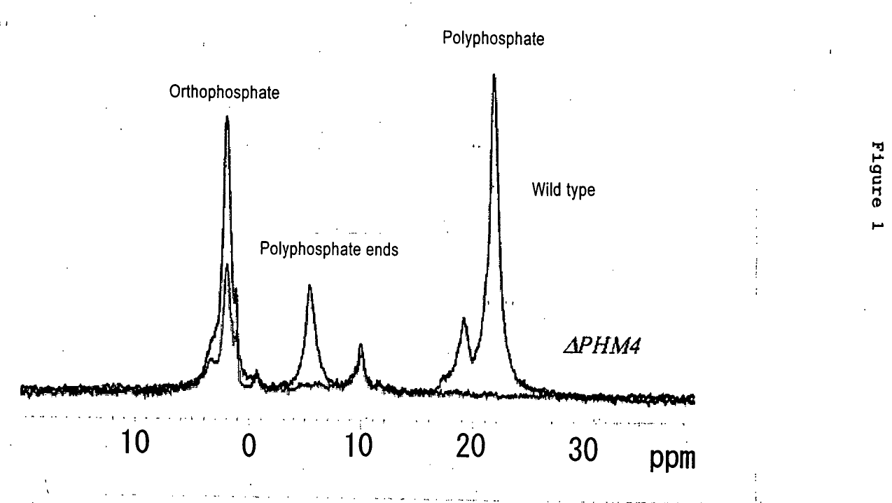

Wild-type line and phm4 knockout line of Saccharomyces cerevisiae (S. cerevislae) were grown on YPD medium (Bacto yeast extract [1% w/v], Bacto peptone [2% w/v]and glucose [2% w/v]), and phosphate (³¹P) NMR spectra of the cell population were quantified by using a DRX-500 MHz spectrometer (Bruker). Using literature values, separate signals corresponding to intracellular orthophosphate, and polyphosphate endo and exo groups were identified. The result is shown in FIG. 1. As is apparent from FIG. 1, the most of phosphate was accumulated as polyphoshpate in wild type yeast cells. However, polyphosphate was not present in the phm4 knockout line.

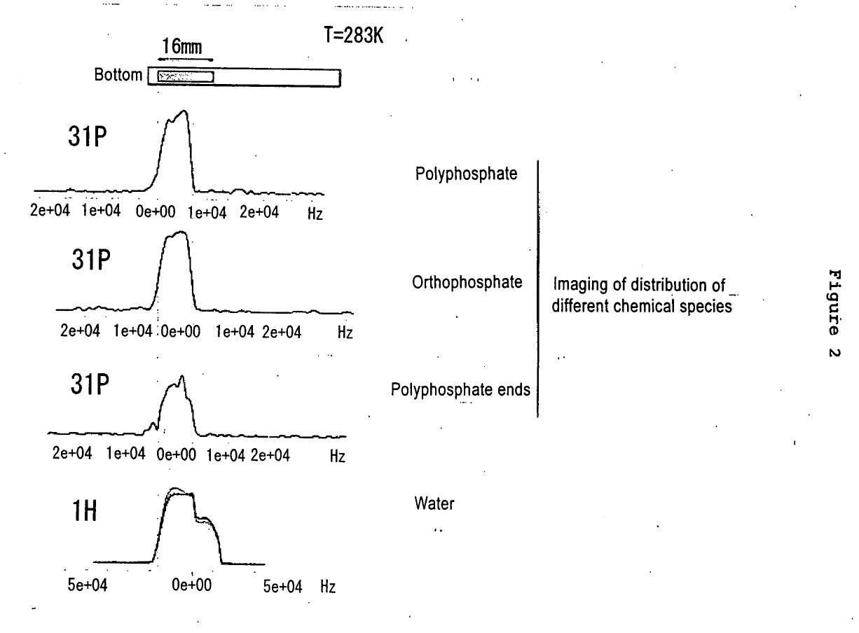

After NMR measuring tubes were filled with cells, one-dimensional imaging (one-dimensional profile) by ³¹P-NMR was taken. The result is shown in FIG. 2. As is apparent from FIG. 2, the profiles could be obtained by using either signal from polyphosphate endo or exo groups, or from orthophosphate. This indicates that imaging of intracellular polyphosphate is possible. On the other hand, it was revealed that one-dimensional profiles using signals derived from water by the frequently used ¹H (hydrogen nuclei) -MRI technique, cannot distinguish the section filled with cells from the section of suspension.

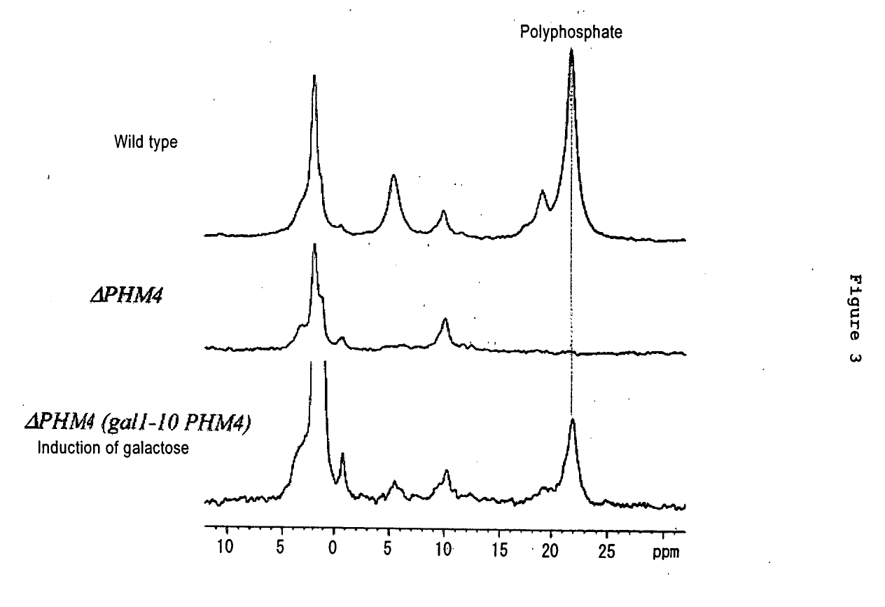

Plasmids carrying PHM4 genes downstream from the GAL1 promoter were used to transform the phm4 knockout line. After transformants were selected on a minimal synthetic medium, they were grown in YPG medium (Bacto yeast extract [1% w/v], Bacto peptone [2% w/v]and galactose [2% w/v]), and the expression of PHM4 genes was induced. The amount of polyphosphate after induction was quantified by the same method as that used in FIG. 1. The result is shown in FIG. 3. As is apparent from FIG. 3, a clear signal due to polyphosphate could be observed. Therefore, the activity of GAL1 promoters could be monitored by ³¹P-NMR signal of polyphosphate.

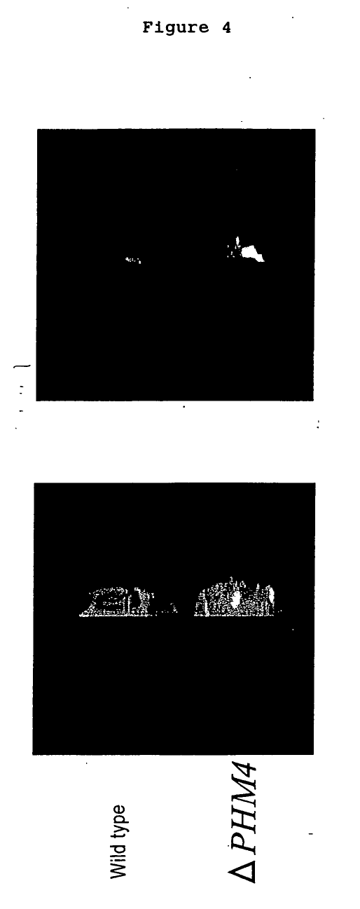

Wild type line (FIG. 4, left) and phm4 knockout line (FIG. 4, right) of Saccharomyces cerevisiae (S. cerevisiae) were cultured on YPD media with 1 mM of FeCl₃, and colonies were swabbed onto an acrylic plate 3 mm in width and 1 mm in thickness, placed in NMR tubes and imaging of colonies from cross section by ¹H-NMR was performed. The result is shown in FIG. 4. These results revealed that equivalent signals could be detected in both yeast strains by a usual NMR imaging method (FIG. 4, lower panel). The influence of iron ions bound to polyphosphate was emphasized by a modified NMR imaging method; the NMR signals were significantly attenuated in wild yeast by the iron ions (FIG. 4, upper panel), as a result of imaging the aforementioned colonies using the specific technique. It is thought to be possible to delete the signals of wild-type yeast strain completely by improving the conditions further. The expression of PHM4 can be monitored very sensitively with this technique of quantification.

### Industrial Applicability

The present invention is a technique by which highly detailed gene expression profiles for analyzing gene function can be obtained safely and conveniently, i.e. a non-destructive and high-resolution versatile visualizing technique, applicable in vivo, and by which the expression level of genes can be monitored in real time, and which can be applied not only to microorganisms and cells but also to deep tissues of mice and the like.

1. A method of monitoring expression of a chosen gene, wherein accumulation of a molecule that varies a NMR signal and can be quantified by NMR, without the requirement to add an exogenous substrate.

2. The method of monitoring an expression of a chosen gene according to claim 1, wherein the molecule that varies the NMR signal and can be quantified by NMR is a polyphosphate.

3. The method of monitoring expression of a chosen gene according to claim 2, wherein the polyphosphate is a polyphosphate generated by expression of a polyphosphate synthetase gene placed downstream of the chosen gene.

4. The method of monitoring expression of a chosen gene according to claim 2, wherein the polyphosphate is a polyphosphate generated by expression of the polyphosphate synthetase gene placed downstream of the chosen gene in-frame.

5. The method of monitoring expression of a chosen gene according to claim 1, wherein the molecule that varies the NMR signal and can be quantified by NMR is a type of cytochrome.

6. The method of monitoring expression of a chosen gene according to any one of claims 1 to 5, wherein the quantification by NMR is a non-destructive quantification by NMR.

7. The method of monitoring expression of a chosen gene according to any one of claims 1 to 6, wherein the expression of the chosen gene is monitored in real time.

8. The method of monitoring expression of a chosen gene according to any one of claims 1 to 7, wherein the expression level of the chosen gene within a cell, a tissue or an organ is detected.

9. The method of monitoring expression of a chosen gene according to any one of claims 1 to 8, wherein the chosen gene is a target gene of a general transcription factor.

10. A method for screening various types of agents, wherein the method of monitoring expression of a chosen gene according to any one of claims 1 to 9 is used.
